(19) United States
(12) Patent Application Publication
(10) Pub. No.: US 2022/0031518 A1
(43) Pub. Date: Feb. 3, 2022

(54) FOOT PATCH

(71) Applicant: Everyday Alchemy, LLC, Brooklyn, NY (US)

(72) Inventor: Austin KATZ, Brooklyn, NY (US)

(73) Assignee: Everyday Alchemy, LLC, Brookly, NY (US)

(21) Appl. No.: 16/983,738

(22) Filed: Aug. 3, 2020

(51) Int. Cl. A61F 13/00; A61F 13/06; A61K 31/05; A61K 31/045; A61K 36/28; A61K 36/185; A61K 9/70

(52) U.S. Cl. CPC A61F 13/00063; A61F 13/067; A61K 31/05; A61K 9/703; A61K 36/28; A61K 36/185; A61K 31/045

(57) ABSTRACT

A foot patch is described herein where the foot patch is configured for placement upon a sole of a foot for sustained delivery of one or more agents. The patch may generally comprise a distal patch body and an elongated proximal portion positioned to project from one end of the distal portion. A proximal patch body may also be included for placement upon a heel of the sole of the foot. The patches may be infused with one or more agents including CBD, arnica oil, and menthol for transdermal delivery into the sole when the patches are adhered to the sole for a period of time.

### FIELD OF THE INVENTION

The present invention relates to methods and apparatus for alleviating foot pain. More particularly, the present invention relates to methods and apparatus for alleviating foot pain via the transdermal application of foot patches infused with one or more agents.

### BACKGROUND OF THE INVENTION

Transdermal patches have typically been used for placement upon the skin of a user in order to provide some therapeutic benefit such as providing treatments, for example, anti-inflammatory, anti-spasmodic, anti-convulsant, etc. Patches are generally applied upon the skin over the affected region for treatment.

Patches have also been placed upon various regions of the foot or feet of a user for treating pain due to conditions such as inflammation, overuse, or injury. These conditions may occur from any number of factors or conditions such as injury, walking or standing, physiological conditions such as plantar fasciitis (inflammation of the plantar fascia), metatarsalgia (inflammation of the ball of the foot), flat feet (fallen arches), etc.

However, because the feet and in particular the soles or plantar aspect of the feet typically bear the weight of a person and are constantly under flexion when walking, running, or standing, adhering patches to the soles or portions of the soles is difficult.

Furthermore, having one or more patches which can provide effective transdermal delivery of agents while remaining in a low profile to accommodate shoes or other footwear is difficult as well.

Accordingly, there exists a need for methods and apparatus for effectively applying one or more patches for the transdermal delivery of agents to the soles of the foot or feet of a user while remaining comfortable during application and use.

### SUMMARY OF THE INVENTION

In order to accommodate and allow for the natural movement of the foot as it flexes and expands when walking, running, climbing, etc., the patches described herein may be separated into two separate components for placement at different locations along the soles or plantar aspect of the foot. Accordingly, one or both components may be selectively applied to a single foot and/or to both feet of the user. The patches may be infused with one or more agents, as described in further detail below, for the sustained transdermal delivery of the agents through the sole or soles of the user's foot or feet.

Because the patch assemblies are separate from one another, the user can apply a single assembly to either the left or right sole or both assemblies may be applied simultaneously to both the left and right soles. Moreover, the different individual components may be applied in any number of combinations as desired depending upon which portions of the user's feet require treatment by patch application. For instance, a first distal patch and a second distal patch may be applied alone to both soles or a first proximal patch and second proximal patch may be applied alone to both soles. In yet another alternative, the first distal patch may be applied to the right sole while the second proximal patch may be applied to the left sole, or the opposite may be applied, or other combinations between the various patch components may be used alone or in combination.

Regardless of the combination of patches used, each patch may be fabricated from a flexible fabric or patch which may retain one or more agents for transdermal delivery of the agents. One example of a flexible fabric or patch may include kinesthesiology fabric patches that are generally 95% cotton and 5% polyester. The patches may be shaped to conform to the sole of a subject's foot. Each of the patches (or a select number of the patches) may be infused with an effective amount of cannabinoid, specifically cannabidiol (CBD), which is one of the most prevalent active ingredients of cannabis (marijuana) and generally considered a non-psychoactive cannabinoid. Alternatively, the patches may be infused with cannabinol (CBN), which is generally considered a mildly psychoactive cannabinoid. One example of a CBD or CBN agent which may be used may include 99% Cannabis sativa seed oil (cannabinol) hemp oil. The CBD or CBN agent may be provided in the patches such that the sustained contact with the skin may facilitate the delivery of the CBD or CBN through the soles and foot or feet of the user for pain relief particularly to the foot or feet as well as potentially providing antibacterial and/or anti-convulsive treatment to the user through delivery via the sole or soles of the feet. The CBD or CBN may be infused within the patches in combination with effective amounts of, e.g., menthol and arnica oil. Other agents which may also be combined with the CBD or CBN may include, e.g., Helianthus annuus (sunflower) seed oil. One example of a combination of agents for infusion into the patches may include, e.g., 99% Cannabis sativa seed oil (cannabinol) hemp oil, Helianthus annuus (sunflower) seed oil, organic arnica oil, and menthol.

The patches may be worn by the user in socks as well as various footwear such as shoes or other footwear and the patches may be fabricated to each have a uniform thickness. Alternatively, different regions of each patch or different patches may be made to have different thickness. Because the patches are adhered along the skin surface of the soles, the contact surfaces of the first patch assembly and the contact surfaces of the second patch assembly may include an adhesive, e.g., water-based acrylic adhesive, as a non-active ingredient and which does not interfere with the transdermal delivery of the agents to the skin. When the patches are stored and not in use, a removable film may be adhered to the contact surfaces. The film may be removed from the patch prior to use to expose the adhesive for placement against the skin surface. With the patches adhering to the skin surfaces of the sole, the wearing of socks or other footwear may also provide for further reinforcement to ensure adherence of the patches to the skin surface. Furthermore, the patches may provide for adequate securement to the skin surface of the sole for a full day of use without falling or peeling off.

In one variation of the patch design, the first distal patch may have an enlarged distal portion which is curved and radiused and tapers at a curvature to extend into an elongated proximal portion which projects along one side of the distal portion and terminates in a curved proximal end. This particular shape of the first patch assembly is configured for adhering to the user's right foot. A first surface may be defined by the patch for contact against the sock, footwear, floor surface, etc. while the opposite second surface or contact surface may come into contact with the skin surface for securement thereto. The distal portion may define a first width where the curvature may begin and the distal portion may define an overall second width. With the elongated proximal portion extending from the distal portion, the portion may taper from a fourth width where it initially extends from the distal portion down to a third width where the proximal portion terminates at a curved proximal end.

The first proximal patch may be formed as a patch component separate from the first distal patch. The proximal patch may be formed as a circularly shaped patch having a diameter and similarly defining a first surface defined by the patch for contact against the sock, footwear, floor surface, etc. while the opposite second surface or contact surface may come into contact with the skin surface for securement thereto. The proximal patch may be sized into different configurations in alternative designs, e.g., elliptical, rectangular, square, pentagonal, hexagonal, etc. so long as the patch is sized to cover the heel of the user.

The second patch assembly may be configured to have the same or similar dimensions but in a mirrored opposite configuration from the first patch assembly so that the second patch assembly may be adhered to the user's opposite foot, e.g., left foot. Also similarly, the second proximal patch may be formed as a patch component separate from the first distal patch.

The patches may be sized to correspond to differently sized feet across various users. For example, the patch assemblies may be sized for small, medium, large, or extra-large feet so that the patch assemblies have standard sizes within specified ranges.

One variation of a transdermal patch which is configured for placement upon a sole of a foot for sustained delivery of one or more agents may generally comprise a distal patch body defining a contact surface for securement to the sole and having a distal portion and an elongated proximal portion positioned to project from one end of the distal portion, wherein the distal portion defines a curved distal border configured for placement proximal to one or more toes of the foot and a curved proximal border configured to extend along or over an anterior transverse arch of the sole such that the distal portion is configured to span a transverse arch of the sole when adhered via the contact surface, wherein the elongated proximal portion defines a length which is configured to extend proximally along and to cover a lateral longitudinal arch of the sole when adhered via the contact surface, and wherein the distal patch body is infused with the one or more agents including CBD, arnica oil, and menthol for transdermal delivery into the sole when the distal patch body is adhered to the sole for a period of time.

One variation of a transdermal patch assembly may generally comprise a first proximal patch body sized for placement upon a first heel of a first sole of a first foot, a first distal patch body separate from the proximal patch body and defining a contact surface for securement to the first sole of the first foot and having a distal portion and an elongated proximal portion positioned to project from one end of the distal portion, wherein the distal portion defines a curved distal border configured for placement proximal to one or more toes of the first foot and a curved proximal border configured to extend along or over an anterior transverse arch of the first sole such that the distal portion is configured to span a transverse arch of the first sole when adhered via the contact surface, wherein the elongated proximal portion defines a length which is configured to extend proximally along and to cover a lateral longitudinal arch of the first sole when adhered via the contact surface, and wherein the first proximal patch body and first distal patch body are infused with one or more agents including CBD, arnica oil, and menthol for transdermal delivery into the first sole when the distal patch body is adhered to the first sole for a period of time.

In one method of treating a foot of a subject, the method may generally comprise applying a distal patch body via a contact surface upon a sole of the foot such that a curved distal border of a distal portion is adhered proximal to one or more toes of the foot and a curved proximal border of the distal portion is adhered to extend along or over an anterior transverse arch of the sole such that the distal portion spans a transverse arch of the sole, further applying the distal patch body such that an elongated proximal portion projecting from one end of the distal portion is adhered to cover a lateral longitudinal arch of the sole, and maintaining the distal patch body upon the sole of the foot for a period of time such that one or more agents including CBD, arnica oil, and menthol which are infused into the distal patch body are delivered transdermally into the sole.

### BRIEF DESCRIPTION OF THE DRAWINGS

FIG. 1A shows a bottom view of a foot patch configured for placement upon the ball and longitudinal arch and also upon the heel of a subject's sole of the right foot.

FIG. 1B shows a bottom view of a foot patch configured for placement upon the ball and longitudinal arch and also upon the heel of a subject's sole of the left foot.

FIG. 2 shows a perspective view of both sets of foot patches.

FIGS. 3A to 3C show bottom, side, and front views of the foot patch configured for placement upon the ball and longitudinal arch of the right foot.

FIGS. 4A to 4C show bottom, side, and front views of the foot patch configured for placement upon the heel of the right foot.

FIGS. 5A to 5C show bottom, side, and front views of the foot patch configured for placement upon the ball and longitudinal arch of the left foot.

FIGS. 6A to 6C show bottom, side, and front views of the foot patch configured for placement upon the heel of the left foot.

FIGS. 7A and 7B illustrate a bottom view of the foot patches adhered to the soles of the feet.

### DETAILED DESCRIPTION OF THE INVENTION

In order to accommodate and allow for the natural movement of the foot as it flexes and expands when walking, running, climbing, etc., the patch may be separated into two separate components for placement at different locations along the soles or plantar aspect of the foot. Accordingly, one or both components may be selectively applied to a single foot and/or to both feet of the user. The patches may be infused with one or more drugs or other agents, as described in further detail below, for the sustained transdermal delivery of the drugs or other agents through the sole or soles of the user's foot or feet

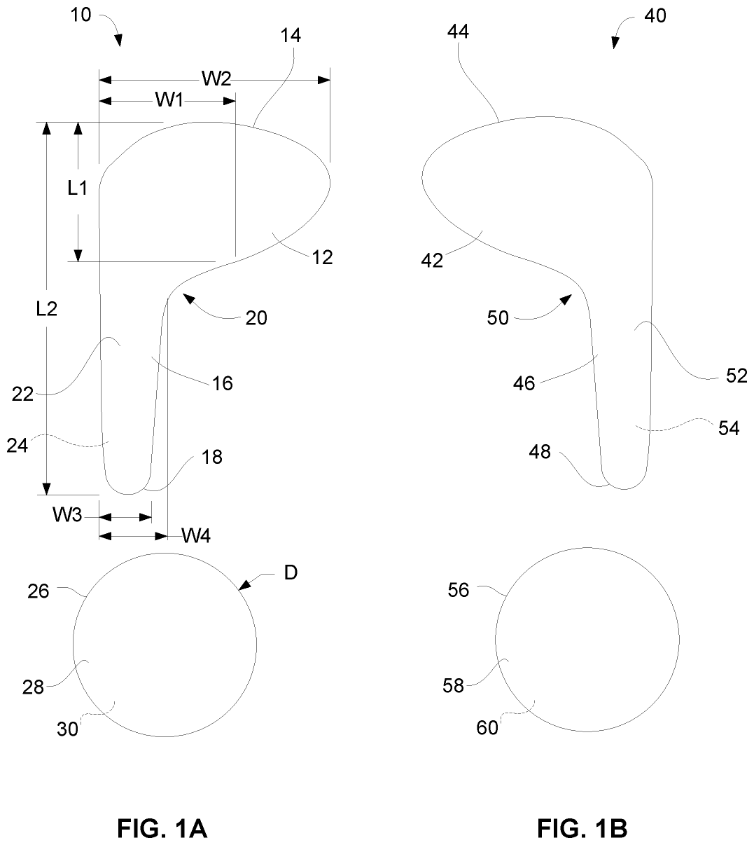

FIG. 1A shows a bottom view of a first patch assembly 10 having two components with a first distal patch 12 and a first proximal patch 26 which are separate from one another and which may be configured for placement upon the sole of a user's right foot. FIG. 1B shows a bottom view of a second patch assembly 40 similarly having two components with a second distal patch 42 and a second proximal patch 56 which are also separate from one another and which maybe configured for placement upon the sole of the user's left foot. Each of the distal patches 12, 42 and proximal patches 26, 56 may be formed of a patch body which are sized as described herein.

Because the patch assemblies 10, 40 are separate from one another, the user can apply a single assembly to either the left or right sole or both assemblies may be applied simultaneously to both the left and right soles. Moreover, the different individual components may be applied in any number of combinations as desired depending upon which portions of the user's feet require treatment by patch application. For instance, the first distal patch 12 and the second distal patch 42 may be applied alone to both soles or the first proximal patch 26 and second proximal patch 56 may be applied alone to both soles. In yet another alternative, the first distal patch 12 may be applied to the right sole while the second proximal patch 56 may be applied to the left sole, or the opposite may be applied, or other combinations between the various patch components may be used alone or in combination.

Regardless of the combination of patches used, each patch may be fabricated from a flexible fabric or patch which may retain one or more agents for transdermal delivery. One example of a flexible fabric or patch may include kinesthesiology fabric patches that are generally 95% cotton and 5% polyester. The patches may be shaped to conform to the sole of a subject's foot. Each of the patches (or a select number of the patches) may be infused with an effective amount of cannabinoid, specifically cannabidiol (CBD), which is one of the most prevalent active ingredients of cannabis (marijuana) and generally considered a non-psychoactive cannabinoid. Alternatively, the patches may be infused with cannabinol (CBN), which is generally considered a mildly psychoactive cannabinoid. One example of a CBD or CBN agent which may be used may include 99% Cannabis sativa seed oil (cannabinol) hemp oil. The CBD or CBN agent may be provided in the patches such that the sustained contact with the skin may facilitate the delivery of the CBD or CBN through the soles and foot or feet of the user for pain relief particularly to the foot or feet as well as potentially providing antibacterial and/or anti-convulsive treatment to the user through delivery via the sole or soles of the feet. The CBD or CBN may be infused within the patches in combination with effective amounts of, e.g., menthol and arnica oil. Other agents which may also be combined with the CBD or CBN may include, e.g., Helianthus annuus (sunflower) seed oil. One example of a combination of agents for infusion into the patches may include, e.g., 99% Cannabis sativa seed oil (cannabinol) hemp oil, Helianthus annuus (sunflower) seed oil, organic arnica oil, and menthol.

The patches may be worn by the user in socks as well as various footwear such as shoes or other footwear and the patches may be fabricated to each have a uniform thickness, e.g., 0.01 to 0.20 in. Alternatively, different regions of each patch or different patches may be made to have different thickness. For example, the proximal patches 26, 56 may be fabricated to have a relatively larger thickness while the distal patches 12, 42 may be fabricated to be relatively thinner than the proximal patches 12, 42 due to the differing locations of placement along the sole. Because the patches are adhered along the skin surface of the soles, the contact surfaces 24, 30 of first patch assembly 10 and contact surfaces 54, 60 of second patch assembly 40 may include an adhesive, e.g., water-based acrylic adhesive, as a non-active ingredient and which does not interfere with the transdermal delivery of the agents to the skin. When the patches are stored and not in use, a removable film may be adhered to the contact surfaces. The film may be removed from the patch prior to use to expose the adhesive for placement against the skin surface. With the patches adhering to the skin surfaces of the sole, the wearing of socks or other footwear may also provide for further reinforcement to ensure adherence of the patches to the skin surface. Furthermore, the patches may provide for adequate securement to the skin surface of the sole for a full day of use without falling or peeling off.

In one variation of the patch design, FIG. 1A shows the first distal patch 12 having an enlarged distal portion 14 which is curved and radiused and tapers at curvature 20 to extend into an elongated proximal portion 16 which projects along one side of the distal portion 14 and terminates in a curved proximal end 18. This particular shape of the first patch assembly 10 is configured for adhering to the user's right foot. A first surface 22 may be defined by the patch 12 for contact against the sock, footwear, floor surface, etc. while the opposite second surface 24 or contact surface may come into contact with the skin surface for securement thereto. The distal portion 14 may define a first width W1, e.g., 1.5 in., but may range anywhere between, e.g., 1.3 to 1.9 in., where the curvature 20 may begin and the distal portion 14 may define an overall second width W2, e.g., 2.5 in., but may range anywhere between, e.g., 2.1 to 3.25 in. The distal portion 14 may also define a first length L1, e.g., 1.5 in., but may range anywhere between, e.g., 1.3 to 2.0 in. With the elongated proximal portion 16 extending from the distal portion 14, the portion 16 may taper from a fourth width W4, e.g., 0.8 in., but may range anywhere between, e.g., 0.74 to 1.09 in., where it initially extends from the distal portion 14 down to a third width W3, e.g., 0.5 in., but may range anywhere between, e.g., 0.5 to 0.67 in., where the proximal portion 16 terminates at curved proximal end 18. The overall length of the distal patch 12 may extend to form a second length L2, e.g., 4.0 in., but may range anywhere between, e.g., 3.5 to 5.3 in.

The first proximal patch 26 may be formed as a patch component separate from the first distal patch 12, as shown. The proximal patch 26 may be formed as a circularly shaped patch having a diameter D, e.g., 2.0 in., but may range anywhere between, e.g., 1.6 to 2.5 in., and similarly defining a first surface 28 defined by the patch 26 for contact against the sock, footwear, floor surface, etc. while the opposite second surface 30 or contact surface may come into contact with the skin surface for securement thereto. The proximal patch 26 may be sized into different configurations in alternative designs, e.g., elliptical, rectangular, square, pentagonal, hexagonal, etc. so long as the patch is sized to cover the heel of the user.

The second patch assembly 40 may be configured to have the same or similar dimensions but in a mirrored opposite configuration from the first patch assembly 10 so that the second patch assembly 40 may be adhered to the user's opposite foot, e.g., left foot. Accordingly, the second distal patch 42 may have a second distal portion 44 may which is curved and radiused and tapers at curvature 50 to extend into a second elongated proximal portion 46 which projects along one side of the distal portion 44 opposite to the first elongated proximal portion 16 and terminates in a curved second proximal end 48. The first surface 52 may be defined by the patch 42 for contact against the sock, footwear, floor surface, etc. while the opposite second surface 54 or contact surface may come into contact with the skin surface for securement thereto.

Also similarly, the second proximal patch 56 may be formed as a patch component separate from the first distal patch 42, as shown. The proximal patch 56 may be formed as a circularly shaped patch and similarly defining a first surface 58 defined by the patch 56 for contact against the sock, footwear, floor surface, etc. while the opposite second surface 60 or contact surface may come into contact with the skin surface for securement thereto. As described above, the lengths and widths of the second patch assembly 40 may be similar to the corresponding lengths and widths of the first patch assembly 10.

While the dimensions above are described for one particular size of the patch assemblies 10, 40, the patches may be sized to correspond to differently sized feet across various users. For example, the patch assemblies 10, 40 may be sized for small, medium, large, or extra-large feet so that the patch assemblies 10, 40 have standard sizes within specified ranges.

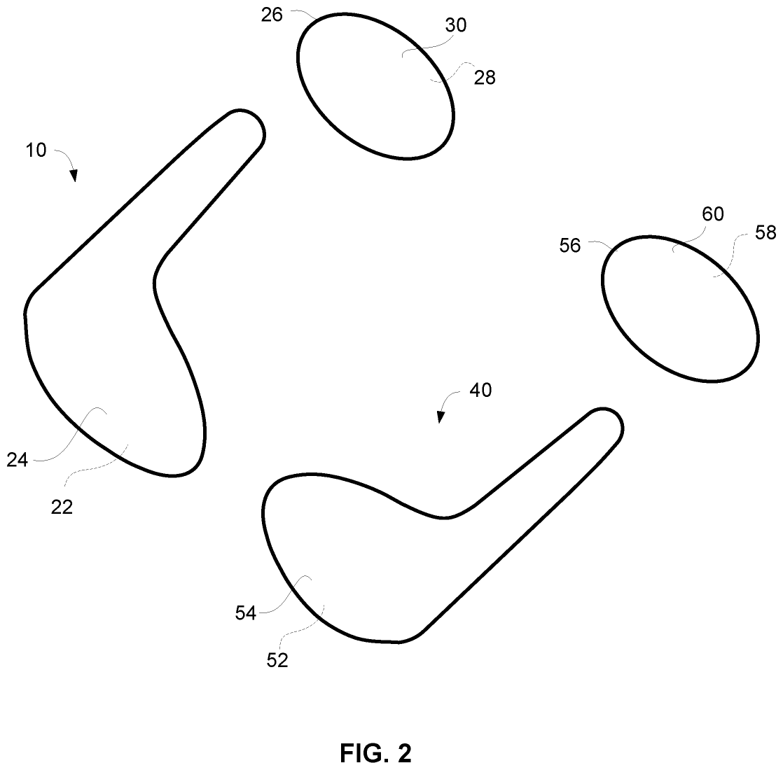

FIG. 2 shows a perspective view of both patch assemblies 10, 40 for illustrative purposes. The various patches are shown with the second surfaces 24, 54 of the distal patches and the second surfaces 30, 60 of the proximal patches 26, 56 exposed for securement against the skin surfaces of the user's feet.

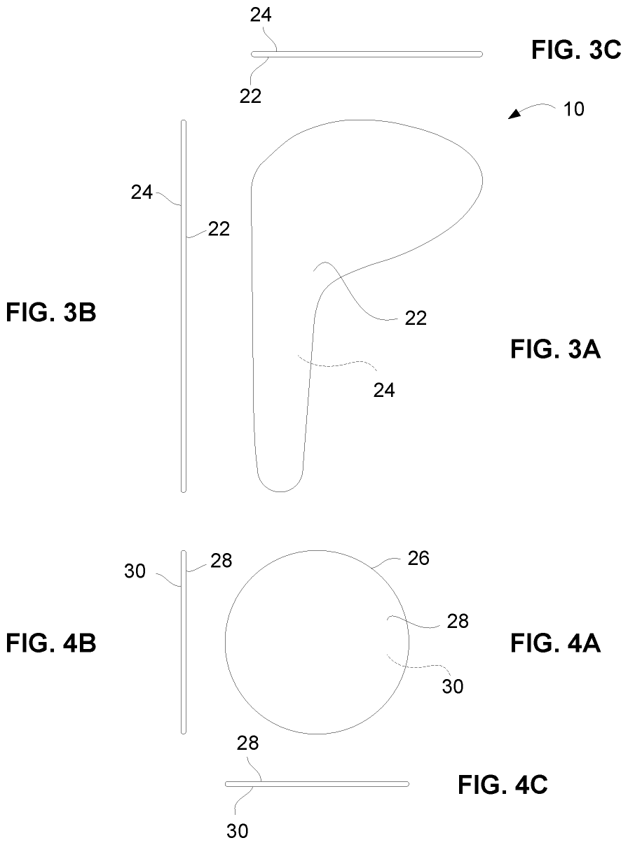

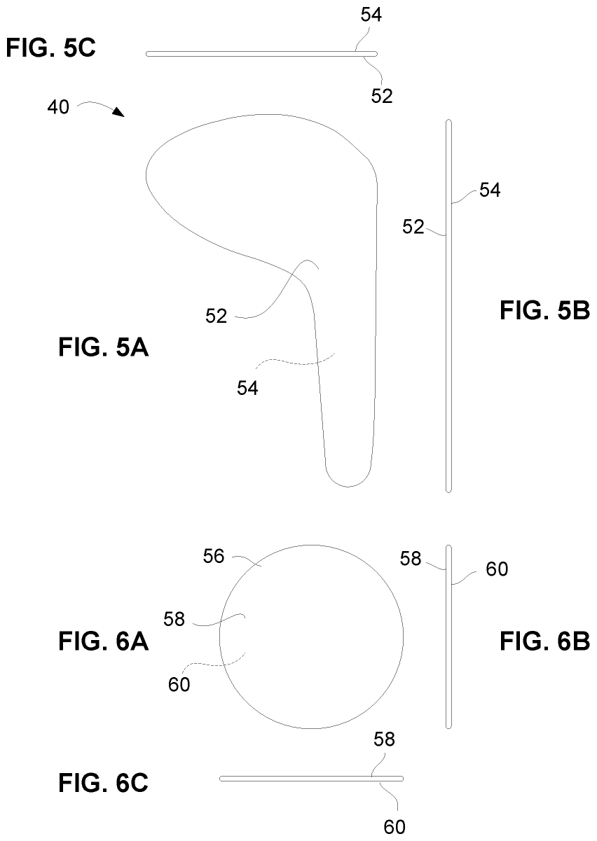

FIGS. 3A to 3C show respective bottom, side, and front views of the first distal patch to illustrate the thickness and FIGS. 4A to 4C show respective bottom, side, and front views of the first proximal patch to illustrate the thickness. Similarly, FIGS. 5A to 5C show respective bottom, side, and front views of the second distal patch to illustrate the thickness and FIGS. 6A to 6C show respective bottom, side, and front views of the second proximal patch to illustrate the thickness.

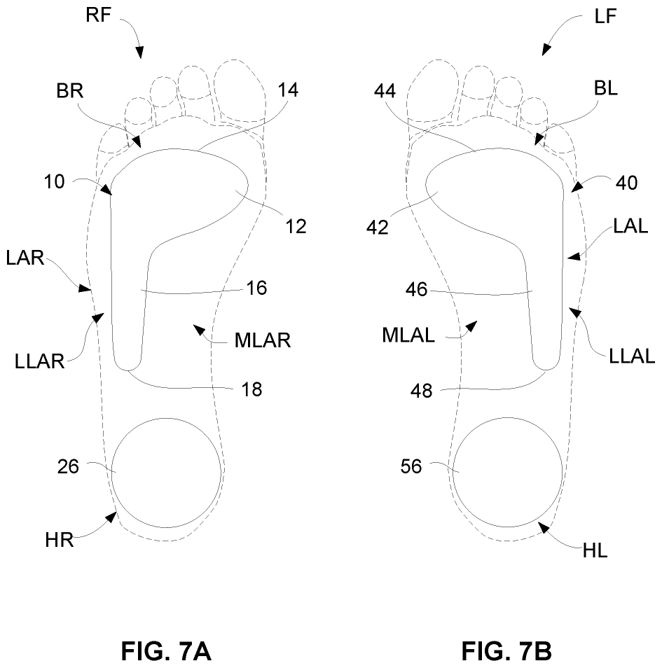

In use when adhered to the sole or soles of the user's foot or feet, a removable film may be removed to expose the securement surfaces and the patch or patches may be applied to the skin surface. FIG. 7A shows one example for how the first patch assembly 10 may be secured to the sole of the right foot RF of a user. Similarly, FIG. 7B shows one example for how the second patch assembly 40 may be secured to the sole of the left foot LF of the user. The first distal patch 12 may be placed so that the first distal portion 14 is secured over the ball BR of the foot RF and span the transverse arch so that the curved distal border of the distal portion 14 is positioned proximal to the toes and the curved proximal border of the distal portion 14 extends along or over the anterior transverse arch. The first elongated proximal portion 16 is positioned to project from the distal portion 14 and extend proximally along and to cover over the longitudinal arch LAR of the right sole, specifically to cover over or along the lateral longitudinal arch LLAR of the right sole. The medial longitudinal arch MLAR may remain exposed and uncovered. The first proximal patch 26 may be adhered over the heel HR of the foot, as shown, so that the first curved proximal end 18 is not contacted and remains separated from the first proximal patch 26. In other variations, the proximal patch 26 may be overlapped with or abut against the first curved proximal end 18.

Similarly, FIG. 7B shows one example for how the second patch assembly 40 may be secured to the sole of the left foot LF of a user. The second distal patch 42 may be placed so that the second distal portion 44 is secured over the ball BL of the foot LF and span the transverse arch so that the curved distal border of the second distal portion 44 is positioned proximal to the toes and the curved proximal border of the second distal portion 44 extends along or over the anterior transverse arch. The second elongated proximal portion 46 is positioned to project from the distal portion 44 and extend proximally along and to cover over the longitudinal arch LAL of the left sole, specifically to cover over or along the lateral longitudinal arch LLAL of the left sole. The medial longitudinal arch MLAL may similarly remain exposed and uncovered. The second proximal patch 56 may be adhered over the heel HL of the foot so that the second curved proximal end 48 is not contacted and remains separated from the second proximal patch 56. In other variations, the second proximal patch 56 may be overlapped with or abut against the second curved proximal end 48.

Although both patch assemblies 10, 40 having both components are illustrated positioned upon both feet, any number of combinations of the patches over one or both feet may be utilized depending upon the desired effect. For example, a single distal patch 12 or 14 or both distal patches 12, 14 may be used alone. Alternatively, a single proximal patch 26 or 56 or both proximal patches 26, 56 may be used alone. In yet another alternative, a single distal patch 12 and a single proximal patch 56 may be used in an alternate pattern on both feet or a single distal patch 42 and a single proximal patch 26 may be used in yet another alternate pattern on both feet.

### Example

In one particular example, the first and second distal patches 12, 42 and first and second proximal patches 26, 56 may both be adhered to the right and left soles of a user, as described herein, so that four individual patches are applied over both feet. Each of the individual patches may be infused with 10 mg of CBD, 4 mg of arnica oil, and 6 mg of menthol. Hence, with two patches applied, for example upon one foot, a total of 20 mg of CBD, 8 mg of arnica oil, and 12 mg of menthol may be delivered transdermally to the user through the sole of one foot over, e.g., an eight-hour period of time. Similarly, with four patches applied, a total of 40 mg of CBD, 16 mg of arnica oil, and 24 mg of menthol may be delivered transdermally to the user through the soles of both feet over, e.g., up to an eight-hour period of time or longer. Any of the individual patches may also be applied alone or in various combinations, e.g., up to an eight-hour period of time or longer, although the amount of the agents which are delivered transdermally will vary depending upon the number of patches used and the amount of time applied. Alternatively, the individual patch or patches may be removed from the sole at any time upon an indication that symptoms of pain are alleviated.

The combination of the CBD, arnica oil, and menthol may work together synergistically to reduce pain to the feet of the user. The CBD may engage the subject's endocannabinoid system, the arnica oil may expedite healing and provide relief from inflammation, while the menthol may generate increased blood flow to the area.

The applications of the devices and methods discussed above are not limited to the described embodiments but may include any number of other configurations. Moreover, such devices and methods may be applied to other treatment sites upon the body where applicable. Modification of the above-described assemblies and methods for carrying out the invention, combinations between different variations as practicable, and variations of aspects of the invention that are obvious to those of skill in the art are intended to be within the scope of the claims.

What is claimed is:

1. A transdermal patch configured for placement upon a sole of a foot for sustained delivery of one or more agents, comprising:
a distal patch body defining a contact surface for securement to the sole and having a distal portion and an elongated proximal portion positioned to project from one end of the distal portion,
wherein the distal portion defines a curved distal border configured for placement proximal to one or more toes of the foot and a curved proximal border configured to extend along or over an anterior transverse arch of the sole such that the distal portion is configured to span a transverse arch of the sole when adhered via the contact surface,
wherein the elongated proximal portion defines a length which is configured to extend proximally along and to cover a lateral longitudinal arch of the sole when adhered via the contact surface, and
wherein the distal patch body is infused with the one or more agents including CBD, arnica oil, and menthol for transdermal delivery into the sole when the distal patch body is adhered to the sole for a period of time.

2. The transdermal patch of claim 1 further comprising a proximal patch body separate from the distal patch body and sized for placement upon a heel of the sole.

3. The transdermal patch of claim 2 wherein the proximal patch body is configured into a circular shape.

4. The transdermal patch of claim 1 wherein the distal patch body is comprised of a fabric having 95% cotton and 5% polyester.

5. The transdermal patch of claim 1 wherein the one or more agents further includes Helianthus annuus (sunflower) seed oil.

6. The transdermal patch of claim 1 wherein the CBD is comprised of 99% Cannabis sativa seed oil hemp oil.

7. The transdermal patch of claim 1 wherein the one or more agents comprise 10 mg of CBD, 4 mg of arnica oil, and 6 mg of menthol.

8. A transdermal patch assembly, comprising:
a first proximal patch body sized for placement upon a first heel of a first sole of a first foot;
a first distal patch body separate from the proximal patch body and defining a contact surface for securement to the first sole of the first foot and having a distal portion and an elongated proximal portion positioned to project from one end of the distal portion,
wherein the distal portion defines a curved distal border configured for placement proximal to one or more toes of the first foot and a curved proximal border configured to extend along or over an anterior transverse arch of the first sole such that the distal portion is configured to span a transverse arch of the first sole when adhered via the contact surface,
wherein the elongated proximal portion defines a length which is configured to extend proximally along and to cover a lateral longitudinal arch of the first sole when adhered via the contact surface, and
wherein the first proximal patch body and first distal patch body are infused with one or more agents including CBD, arnica oil, and menthol for transdermal delivery into the first sole when the distal patch body is adhered to the first sole for a period of time.

9. The transdermal patch assembly of claim 8 further comprising a second proximal patch body sized for placement upon a second heel of a second sole of a second foot and a second distal patch body separate from the second proximal patch body and defining a contact surface for securement to the second sole of the second foot.

10. The transdermal patch assembly of claim 8 wherein the first proximal patch body and first distal patch body are comprised of a fabric having 95% cotton and 5% polyester.

11. The transdermal patch assembly of claim 8 wherein the one or more agents further includes Helianthus annuus (sunflower) seed oil.

12. The transdermal patch assembly of claim 8 wherein the CBD is comprised of 99% Cannabis sativa seed oil hemp oil.

13. The transdermal patch assembly of claim 8 wherein the one or more agents comprise 20 mg of CBD, 8 mg of arnica oil, and 12 mg of menthol.

14. A method of treating a foot of a subject, comprising:
applying a distal patch body via a contact surface upon a sole of the foot such that a curved distal border of a distal portion is adhered proximal to one or more toes of the foot and a curved proximal border of the distal portion is adhered to extend along or over an anterior transverse arch of the sole such that the distal portion spans a transverse arch of the sole;
further applying the distal patch body such that an elongated proximal portion projecting from one end of the distal portion is adhered to cover a lateral longitudinal arch of the sole; and
maintaining the distal patch body upon the sole of the foot for a period of time such that one or more agents including CBD, arnica oil, and menthol which are infused into the distal patch body are delivered transdermally into the sole.

15. The method of claim 14 further comprising applying a proximal patch body via a contact surface upon a heel of the sole, wherein the proximal patch body is separate from the distal patch body.

16. The method of claim 15 further comprising maintaining the proximal patch body upon the heel for a period of time such that the one or more agents which are infused into the proximal patch body are delivered transdermally into the heel.

17. The method of claim 15 further comprising applying a second distal patch body defining a second contact surface upon a second sole of a second foot.

18. The method of claim 17 further comprising applying a second proximal patch body via a second contact surface upon a second heel of the second foot, wherein the second proximal patch body is separate from the second distal patch body.

19. The method of claim 14 wherein maintaining the distal patch body comprises maintaining the distal patch body for up to an eight-hour period of time.

20. The method of claim 14 further comprising removing a film from the contact surface prior to applying the distal patch body.
